(19) United States
(12) Patent Application Publication
(10) Pub. No.: US 2024/0307085 A1
(43) Pub. Date: Sep. 19, 2024

(54) SURGICAL TOOLS WITH OCCLUDED BLADES

(71) Applicant: Cilag GmbH International, Zug (CH)

(72) Inventor: Pablo Hambly, San Jose, CA (US)

(21) Appl. No.: 18/121,326

(22) Filed: Mar. 14, 2023

(51) Int. Cl. A61B 17/295

(52) U.S. Cl. CPC A61B 17/295; A61B 2017/2926; A61B 2017/2947; A61B 2034/302

(57) ABSTRACT

Surgical tools that are capable of both manipulating a needle and cutting surgical thread during a suturing operation can sometimes be problematic due to an inability to sever the suture during a suturing operation. Surgical tools and end effectors, which are designed to secure the suture within the cutting region of the end effector until severed and then allow the suture to be freely released can substantially mitigate this issue. Such surgical tools may comprise an end effector with a first and second jaw with an incorporated cutting body. The cutting body of the first and second jaw may have a substantially flat portion followed by a curved concave and convex portion that is designed to trap the surgical thread within the cutting portion.

### TECHNICAL FIELD

The systems and methods disclosed herein are directed to robotic surgical tools and, more particularly to, articulable surgical tools.

### BACKGROUND

Minimally invasive surgical (MIS) tools and procedures can often be preferred over traditional open surgical techniques due to their ability to decrease post-operative recovery time and to leave minimal scarring. Laparoscopic surgery is one type of MIS procedure in which one or more small incisions are formed in the abdomen of a patient and a trocar is inserted through each incision to provide a surgical access pathway for an appropriate surgical tool. Trocars can additionally provide an internal seal assembly for maintaining insufflation of the abdomen during a surgical procedure.

A variety of MIS tools can be inserted into the abdominal cavity of a patient via a trocar and maneuvered from outside the abdomen. Laparoscopic surgical tools, for example, are often similar to those used in traditional surgical procedures, with the exception that laparoscopic surgical tools possess an elongate shaft extending from an end effector to a location outside the abdomen. The end effector is the surgically functional part of the surgical tool. The elongate shaft protrudes externally through a trocar when the surgical tool is inserted in the abdomen of a patient, and an external portion of the surgical tool provides a means for manipulating and communicating with the end effector. Once inserted in a patient's body, the end effector can engage and/or treat tissue in a number of ways to achieve a desired diagnostic or therapeutic effect. Illustrative end effectors of laparoscopic and similar surgical tools include, for example, scissors, graspers, needle drivers, clamps, staplers, cauterizers, suction tools, irrigation tools, and clip-appliers.

Robotic surgery represents a specialized class of laparoscopic surgical procedures. Instead of directly engaging a surgical tool as in traditional laparoscopic surgery, a surgeon instead manipulates and engages the surgical tool using an electronic interface communicatively coupled to a robotic manipulator. Manipulation and engagement of a surgical tool under robotic control can allow much more precise surgical procedures to be performed in many instances. A surgeon need not necessarily even be in the operating room with the patient. Advantageously, robotic surgical systems can allow intuitive hand movements to be realized by maintaining a natural eye-hand axis. In addition, robotic surgical systems can incorporate a “wrist” coupling the end effector to the elongate shaft to provide natural, hand-like articulation during a robotic surgical procedure. The wrist can also facilitate an expanded and more complex range of motion than is possible with a human wrist, which can allow highly elaborate and precise surgical procedures to be performed.

Many laparoscopic and robotic surgical procedures utilize an end effector that is capable of performing a suturing operation. As in conventional surgical procedures, laparoscopic and robotic suturing operations utilize a needle attached to a length of surgical thread for placing one or more sutures in a tissue. Laparoscopic and robotic suturing operations utilize a needle driver as the end effector for manipulating the needle when placing sutures. The needle driver comprises opposing jaws that articulate between closed and open positions when grasping and releasing the suturing needle. Upon completion of a suturing operation, the surgical thread must be severed (cut) to remove the needle and excess surgical thread from the patient.

Bladed cutting instruments such as surgical scissors or shears are commonly used to sever surgical thread during surgical procedures. In laparoscopic and robotic surgical procedures, such cutting instruments can be included on the same surgical tool as a needle driver but may also form part of a separate surgical tool. Both approaches can be problematic. Surgical threads can be lightweight and small in diameter, which allows for them to be easily moved when an external force is applied to it. This characteristic could cause the thread to get squeezed by the cutting instrument and moved along the blade cutting surface until it is pushed off the cutting surface or out the end of the instrument. In some instances, when a surgical thread has successfully been severed, the severed end of the thread may become trapped between the opposed blades of the cutting instrument leading to a delay in the surgical procedure or suture failure.

Minimally invasive surgical (MIS) tools and procedures can often be preferred over traditional open surgical techniques due to their ability to decrease post-operative recovery time and to leave minimal scarring. Laparoscopic surgery is one type of MIS procedure in which one or more small incisions are formed in the abdomen of a patient and a trocar is inserted through each incision to provide a surgical access pathway for an appropriate surgical tool. Trocars can additionally provide an internal seal assembly for maintaining insufflation of the abdomen during a surgical procedure.

A variety of MIS tools can be inserted into the abdominal cavity of a patient via a trocar and maneuvered from outside the abdomen. Laparoscopic surgical tools, for example, are often similar to those used in traditional surgical procedures, with the exception that laparoscopic surgical tools possess an elongate shaft extending from an end effector to a location outside the abdomen. The end effector is the surgically functional part of the surgical tool. The elongate shaft protrudes externally through a trocar when the surgical tool is inserted in the abdomen of a patient, and an external portion of the surgical tool provides a means for manipulating and communicating with the end effector. Once inserted in a patient's body, the end effector can engage and/or treat tissue in a number of ways to achieve a desired diagnostic or therapeutic effect. Illustrative end effectors of laparoscopic and similar surgical tools include, for example, scissors, graspers, needle drivers, clamps, staplers, cauterizers, suction tools, irrigation tools, and clip-appliers.

Robotic surgery represents a specialized class of laparoscopic surgical procedures. Instead of directly engaging a surgical tool as in traditional laparoscopic surgery, a surgeon instead manipulates and engages the surgical tool using an electronic interface communicatively coupled to a robotic manipulator. Manipulation and engagement of a surgical tool under robotic control can allow much more precise surgical procedures to be performed in many instances. A surgeon need not necessarily even be in the operating room with the patient. Advantageously, robotic surgical systems can allow intuitive hand movements to be realized by maintaining a natural eye-hand axis. In addition, robotic surgical systems can incorporate a “wrist” coupling the end effector to the elongate shaft to provide natural, hand-like articulation during a robotic surgical procedure. The wrist can also facilitate an expanded and more complex range of motion than is possible with a human wrist, which can allow highly elaborate and precise surgical procedures to be performed.

Many laparoscopic and robotic surgical procedures utilize an end effector that is capable of performing a suturing operation. As in conventional surgical procedures, laparoscopic and robotic suturing operations utilize a needle attached to a length of surgical thread for placing one or more sutures in a tissue. Laparoscopic and robotic suturing operations utilize a needle driver as the end effector for manipulating the needle when placing sutures. The needle driver comprises opposing jaws that articulate between closed and open positions when grasping and releasing the suturing needle. Upon completion of a suturing operation, the surgical thread must be severed (cut) to remove the needle and excess surgical thread from the patient.

Bladed cutting instruments such as surgical scissors or shears are commonly used to sever surgical thread during surgical procedures. In laparoscopic and robotic surgical procedures, such cutting instruments can be included on the same surgical tool as a needle driver but may also form part of a separate surgical tool. Both approaches can be problematic. Surgical threads can be lightweight and small in diameter, which allows for them to be easily moved when an external force is applied to it. This characteristic could cause the thread to get squeezed by the cutting instrument and moved along the blade cutting surface until it is pushed off the cutting surface or out the end of the instrument. In some instances, when a surgical thread has successfully been severed, the severed end of the thread may become trapped between the opposed blades of the cutting instrument leading to a delay in the surgical procedure or suture failure.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 shows a diagram of an illustrative surgical tool that may incorporate certain principles of the present disclosure.

FIG. 2 shows a diagram illustrating the degrees of freedom through which a wrist of a surgical tool may articulate.

FIGS. 3-5 show various views of an illustrative surgical tool containing an end effector.

FIG. 6 shows a diagram illustrating coupling between a surgical tool and a robotic manipulator.

FIG. 7 shows an isometric of an illustrative surgical tool incorporating a needle driver and a bladed cutting instrument.

FIGS. 8 and 9 show an end effector of an illustrative surgical tool with an integrated cutting feature.

FIG. 10 shows a cross-section of an end effector of an illustrative surgical tool with an integrated cutting feature.

FIG. 11 shows an illustrative surgical tool that may incorporate certain principles of the present disclosure.

FIG. 12 is a zoomed-in cross-section of an end effector of an illustrative surgical tool with an integrated cutting feature.

FIGS. 13-16 shows an operational sequence of an end effector of an illustrative surgical tool with an integrated cutting feature severing a surgical thread.

### DETAILED DESCRIPTION

The present disclosure generally describes surgical tools having an end effector operatively coupled to an elongated shaft and, more specifically, surgical tools and end effectors that are capable of both placing sutures and severing surgical thread during a surgical procedure.

Delay in the surgical procedure due to the inability to sever the suture during a suturing operation can be problematic. Likewise, the inability to grasp and control the suture once severed during a suturing operation can also be problematic. The present disclosure describes surgical tools and end effectors that are configured to perform multiple aspects of a suturing operation and methods for use thereof, but with a significantly lower risk of delaying the surgical procedure or experiencing suture failure. More specifically, the present disclosure describes surgical tools and end effectors incorporating both a needle driver and a bladed cutting feature, which are designed to secure the suture within the cutting region of the end effector until severed and then allow the suture to be freely released once severed. As such, the surgical tools and end effectors disclosed herein are much less susceptible to failure to sever or post-sever binding of the suture during a suturing operation.

Before discussing additional details of the surgical tools and end effectors of the present disclosure and methods for their use, a brief overview of laparoscopic and similar surgical tools and robotic surgical systems will be provided hereinafter in order for the embodiments of the present disclosure to be better understood.

The terms “proximal” and “distal” are defined herein relative to the location of engagement by a surgeon or a robotic manipulator. The term “proximal” refers to a position closer to the location of engagement (i.e., further away from a patient), and the term “distal” refers to a position more removed from the location of engagement (i.e., nearer to a patient). Moreover, directional terms such as above, below; upper, lower, upward, downward, left, right, and the like are used to describe relative position in the figures and thus should not be considered limiting.

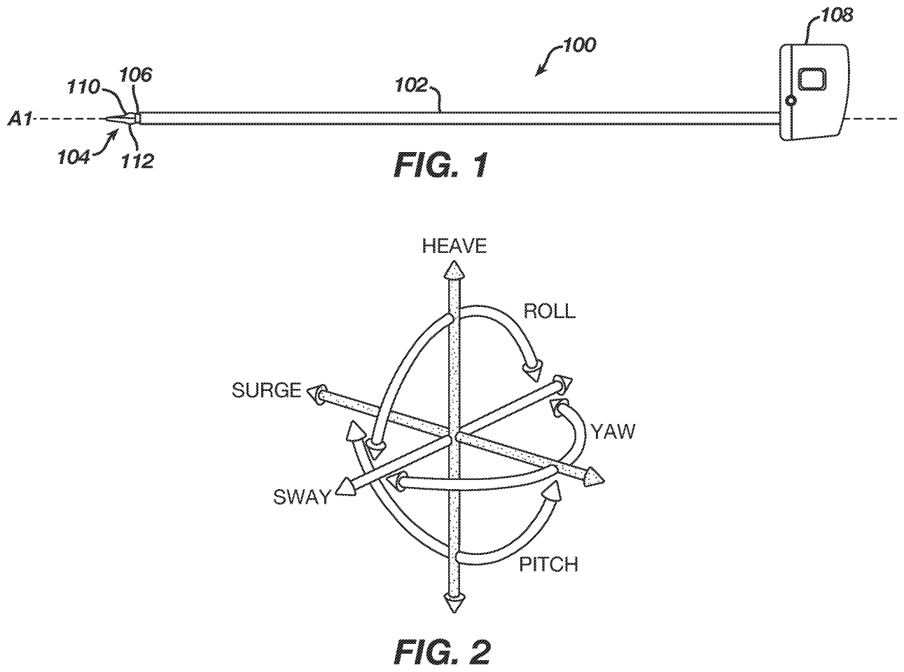

FIG. 1 shows a diagram of an illustrative surgical tool 100 that may incorporate certain principles of the present disclosure. Surgical tool 100 includes elongate shaft 102, end effector 104 located at a distal end of elongate shaft 102, and housing 108 located at a proximal end of elongate shaft 102. Wrist 106 is also located at a distal end of elongate shaft 102 and couples end effector 104 thereto. Housing 108 may be configured for releasable coupling with a mounting fixture of a robotic manipulator, alternately referred to as a “robot” or “surgical robot.” Housing 108 contains various mechanisms (obscured in FIG. 1) which may be actuated to produce one or more resultant motions in end effector 104. More particularly, actuation within housing 108 controls the operation of end effector 104 via retraction and extension of cables or similar elongate members (obscured in FIG. 1) that are operably engaged with end effector 104.

Housing 108 may be releasably coupled with the mounting fixture of a robotic manipulator in a variety of ways, such as by clamping or clipping thereto or slidably mating therewith. Illustrative mechanisms for releasably coupling housing 108 to a mounting fixture are described in more detail in U.S. Patent Application Publications 2015/0209965, 2018/0000543, and 2015/0025549 are incorporated herein by reference in their entirety. Illustrative robotic surgical systems are also described in these references as well as in U.S. Pat. No. 8,831,782, which is also incorporated herein by reference in its entirety.

Continuing with FIG. 1, end effector 104 is configured to move relative to elongate shaft 102 at wrist 106, such as by pivoting at wrist 106, to position end effector 104 at a desired orientation and location relative to a surgical site during a surgical procedure. Housing 108 includes various components designed to position and operate various features of end effector 104 (e.g., one or more of clamping, firing, rotation, articulation, energy delivery, and the like). In illustrative embodiments, one or more elongate members extend from housing 108 through wrist 106 to facilitate articulation of end effector 104, as discussed in more detail herein. Elongate shaft 102 and end effector 104 coupled distally thereto are configured to rotate about longitudinal axis A1. Various components of housing 108 can be configured to facilitate rotational motion of elongate shaft 102 and end effector 104 about longitudinal axis A1. In other embodiments, elongated shaft 102 may be fixed to housing 108, in which case surgical tool 100 may be rotated by the robotic manipulator to reposition elongated shaft 102 and end effector 104.

Surgical tool 100, particularly at end effector 104, can be configured to perform at least one surgical function. The choice of end effector 104 can determine which surgical function surgical tool 100 is able to perform. Illustrative configurations of end effector 104 that may be present in surgical tool 100 include, for example, forceps, graspers, needle drivers, scissors, electrocauterization tools that apply energy to tissue, staplers, clip appliers, suctioning tools, irrigation tools, imaging devices (e.g., endoscopes or ultrasonic probes), and any combination thereof. In at least one embodiment, surgical tool 100 may be configured to apply mechanical force to a tissue. The mechanical force can be conveyed to end effector 104 via the cables or similar elongate members extending through elongate shaft 102.

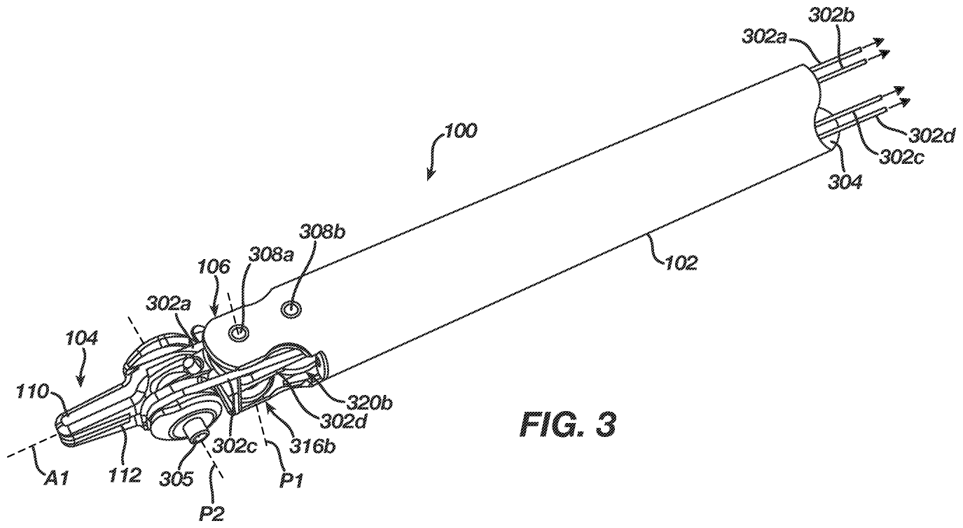

Elongate shaft 102 extends distally from housing 108 and has at least one lumen (see FIG. 3) extending internally therethrough. Elongate shaft 102 may be affixed to housing 108, but alternately may be releasably coupled so as to be interchangeable with other types of elongate shafts, such as elongate shafts may have a differing diameter. Elongate shaft 102 may also be rotatably coupled to housing 108.

End effector 104 can have a variety of sizes, shapes and configurations. In the illustrative configuration of FIG. 1, end effector 104 comprises a tissue grasper or needle driver having opposing jaws 110 and 112 that are configured to move (pivot) relative to one another between open and closed positions. In addition, the entirety of end effector 104 may pivot relative to elongated shaft 102 at wrist 106. Pivoting may place end effector 104 in a desired position to engage tissue or another surface during a surgical procedure.

Wrist 106 can likewise have a variety of configurations. In the illustrative configuration of FIG. 1, wrist 106 includes a joint configured to allow movement of end effector 104 relative to elongate shaft 102, such as a pivot joint at which jaws 110 and 112 are pivotally attached via a corresponding body. Illustrative configurations that may be similar to wrist 106 and are suitable for use in the embodiments of the present disclosure include those described in U.S. Patent Application Publications 2015/0209965, 2018/0000543, and 2015/0025549, each previously incorporated by reference above.

FIG. 2 shows a diagram illustrating the degrees of freedom through which wrist 106 may articulate. More specifically, the degrees of freedom available to wrist 106 are represented by three translational or position variables (e.g., surge, heave and sway) and three rotational or orientation variables (e.g., Euler angles or roll, pitch and yaw). The translational and rotational variables collectively describe the position and orientation of one or more components of a surgical system (e.g., wrist 106 and associated end effector 104) with respect to a given frame of reference, such as a Cartesian coordinate system or spherical coordinate system. As illustrated in FIG. 2, the term “surge” refers to forward and backward movement, the term “heave” refers to up and down movement, and the term “sway” refers to left and right movement. With regard to the rotational terms in FIG. 2, “roll” refers to side-to-side tilting, “pitch” refers to forward and backward tilting, and “yaw” refers to left and right turning.

A pivoting motion can include pitch movement about a first axis of wrist 106 (e.g., X-axis), yaw movement about a second axis of wrist 106 (e.g., Y-axis), and combinations thereof to allow for 360° rotational movement of end effector 104 about wrist 106. The pivoting motion may also be limited to movement in a single plane such that end effector 104 rotates only in a single plane (e.g., only pitch movement about a first axis of wrist 106 or only yaw movement about a second axis of wrist 106).

Surgical tool 100 includes a plurality of cables or similar elongate members (obscured in FIG. 1), which are configured to impart movement to end effector 104 relative to elongate shaft 102. Illustrative forms of the elongate members include, for example, cables, bands, lines, cords, wires, ropes, strings, twisted strings and the like. Elongate members can be formed from any of a variety of high-durability materials, such as a metal (e.g., tungsten, stainless steel, and like materials) or a polymer. One or more of the elongate members may be made of a flexible material. Illustrative cables and similar elongate members are described in U.S. Patent Application Publications 2015/0209965 and 2015/0025549, each previously incorporated herein by reference.

The disposition of the elongate members within surgical tool 100 is illustrated more fully in FIGS. 3-5, which show various enlarged views of elongate shaft 102, end effector 104, and wrist 106. Although surgical tool 100 is depicted as including four elongate members 302a-d, one pair being operatively coupled to each of jaws 110 and 112, alternative configurations can have differing numbers of elongate members. For example, a surgical tool having an end effector that does not require internal motion can include two elongate members configured to provide articulation upon longitudinal tensioning and de-tensioning.

As shown in FIGS. 3-5, elongate members 302a-d extend longitudinally within lumen 304 of elongate shaft 102 through wrist 106 and operably engage end effector 104, as described hereinafter. The proximal ends of elongate members 302a-d are similarly operably engaged with components in housing 108 (not shown in FIGS. 3-5). One or more of elongate members 302a-d may be selectively translated longitudinally to cause end effector 104 to move (e.g., pivot in one or more locations) relative to elongate shaft 102. Depending on the required motion, one or more of elongate members 302a-d may translate longitudinally to articulate end effector 104 (e.g., to move jaws 110 and 112 at an angle in the same direction), to open end effector 104 (e.g., to move jaws 110 and 112 away from one another), to close end effector 104 (e.g., to move jaws 110 and 112 toward one other), or any combination thereof.

Although a single lumen 304 is depicted in FIG. 3, multiple lumens can be present in alternative embodiments, such that one or more of elongate members 302a-d are housed within each of the multiple lumens. In further alternative embodiments, one or more of elongate members 302a-d can extend along the exterior of elongate shaft 102, such as in longitudinal channels formed in an exterior surface of elongate shaft 102.

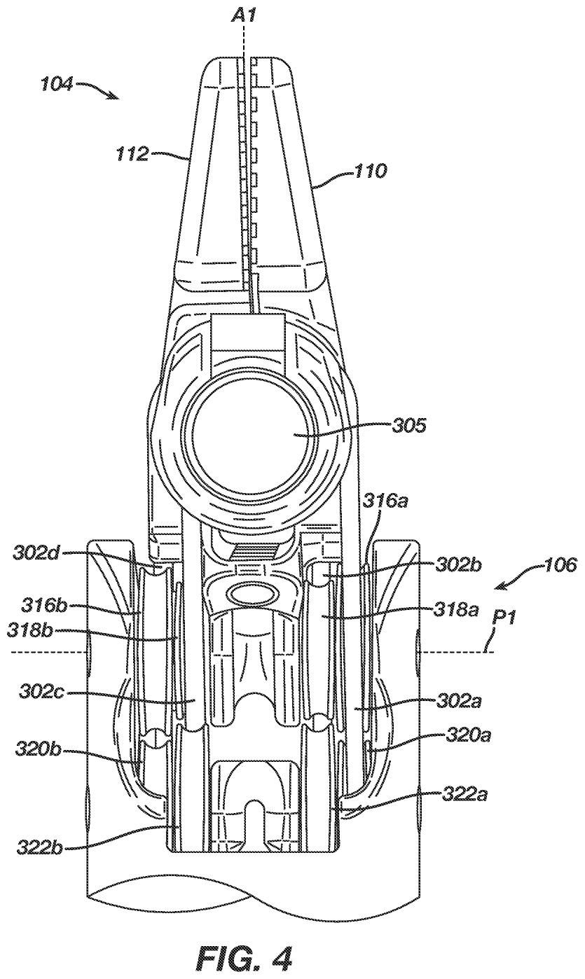

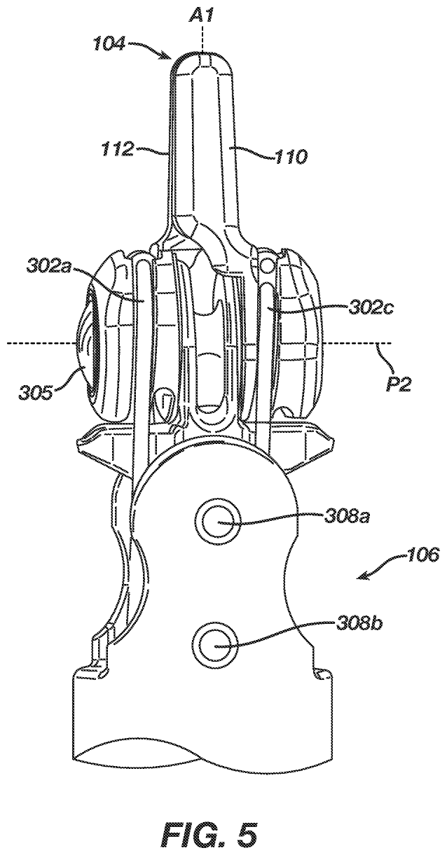

Referring still to FIG. 3, and with further reference to FIGS. 4 and 5, wrist 106 includes multiple pulleys for engaging and redirecting elongate members 302a-d during their longitudinal translation. Specifically, wrist 106 includes distal plurality of pulleys 316a, 316b, 318a and 318b, and proximal plurality of pulleys 320a, 320b, 322a and 322b. Clearance (best shown in FIG. 4) is provided between corresponding pulleys in the distal and proximal pluralities of pulleys, which is sized for passage of elongate members 302a-d therethrough. Pulleys 316a. 316b, 318a and 318b are mounted to distal wrist axle 308a, and pulleys 320a. 320b, 322a and 322b are mounted to proximal wrist axle 308b. End effector 104 is operably coupled to wrist 106 such that distal wrist axle 308a defines first pivot axis P1 during operation thereof.

Surgical tool 100 further includes second pivot axis P2 along end effector axle 305, about which jaws 110 and 112 are configured to pivot relative to each other from a closed position through a range of open positions, and/or about which jaws 110 and 112 are configured to move together during articulation of end effector 104. As illustrated, second pivot axis P2 is substantially perpendicular to longitudinal axis A1. Axes A1 and P2 may not be precisely perpendicular to one another but nevertheless be considered to be substantially perpendicular due to any number of factors, such as manufacturing tolerance and precision of measurement devices.

Surgical tool 100 has two joints at second pivot axis P2, one joint for each of jaws 110 and 112. Actuation of at least one of elongate members 302a-d causes movement of jaw 110 and/or jaw 112 at the associated joint(s) along second pivot axis P2. Jaws 110 and 112 may be configured to pivot in tandem at their associated joints. That is, during opening of jaws 110 and 112, each of jaws 110 and 112 rotates at its associated joint, and during closing of jaws 110 and 112, each of jaws 110 and 112 rotates in the opposite direction at its associated joint.

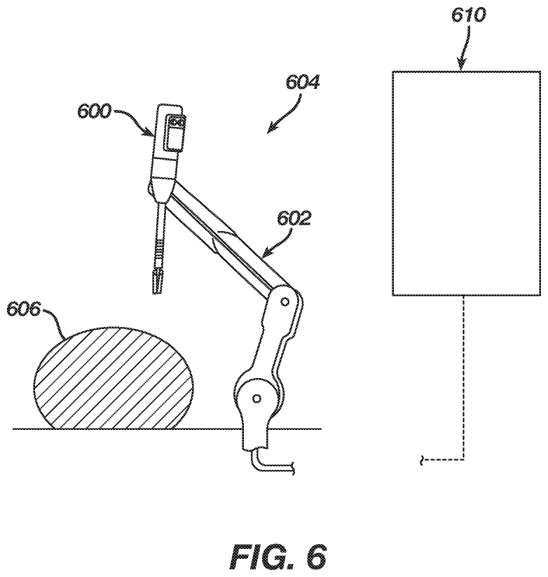

Surgical tool 100 may be configured for releasable coupling to a robotic manipulator. FIG. 6 shows a diagram illustrating coupling between a surgical tool and a robotic manipulator. The manner of coupling depicted in FIG. 6 is illustrative in nature so that certain embodiments of the present disclosure can be better understood. In non-limiting variations, the type of surgical tool and/or robotic manipulator, and/or the manner of coupling, for example, may differ based upon considerations that will be familiar to one having ordinary skill in the art.

As depicted in FIG. 6, surgical tool 600 is coupled to arm 602 of robotic manipulator 604. Robotic manipulator 604 and surgical tool 600 are positioned adjacent to patient 606 in order to conduct a surgical procedure thereon. Robotic manipulator 604 is in electronic communication with control system 610, through which a surgeon may move arm 602 and/or actuate surgical tool 600 according to one or more embodiments. Although FIG. 6 has depicted a wired connection between surgical tool 600 and control system 610, wireless configurations also reside within the scope of the present disclosure. In one or more embodiments, control system 610 may include vision control, processing control, or any combination thereof, using any combination of software and hardware implementation.

As discussed previously, surgical tools conventionally configured both to manipulate a needle during a suturing operation and to sever surgical thread upon completion of the suturing operation can be problematic due to failing to sever the thread or binding of the surgical thread with the surgical instrument. Accordingly, the present disclosure provides end effectors for a surgical tool that are capable of grasping and releasing a needle (or possibly tissue) during suturing and subsequently severing surgical thread upon completing a surgical procedure, but with a much lower risk of procedural delay due to failing to sever or release the surgical thread post severance. More specifically, the end effectors described herein incorporate both a needle driver and cutting region, where the cutting region is shaped to trap the surgical thread. Near the cutting region is a recessed region that releases the thread after cutting. The end effector configuration disclosed herein advantageously offers a lower incidence of failed severance and binding of the surgical thread compared to otherwise comparably equipped conventional dual-function end effectors.

The end effectors described herein advantageously incorporate both needle-driving and thread-severing capabilities within the footprint of a single end effector, thus providing compatibility with other types of laparoscopic and robotic surgical equipment and procedures. More specifically, the end effectors described herein include opposing jaws capable of opening and closing to grasp and release tissue, surgical thread, needles, and the like. As described in further detail herein, the opposing jaws can be further manipulated by opening beyond a predetermined angle to expose blades for cutting surgical thread when desired, such as upon the completion of suturing. Specifically, the opposing jaws can be opened beyond a predetermined angle sufficient to expose blades located distal to a pivot joint of the opposing jaws upon the needle driver. The blades are then ready to receive and sever surgical thread upon at least partially closing the jaws. As such, the end effectors of the present disclosure can facilitate multiple aspects of a suturing operation, particularly needle grasping/releasing and surgical thread severance. Illustrative end effectors with a combined needle driver and cutting features are described in U.S. Patent Application Publication 2019/0105032, which is incorporated herein by reference.

The end effectors described herein are configured such that from a fully closed jaw position up to the predetermined angle, the blades overlap and provide no aperture (gap) into which surgical thread can be received. As such, the blades are effectively occluded, except when the severing of surgical thread is desired. Further advantages of the end effectors and surgical tools of the present disclosure are provided hereinbelow.

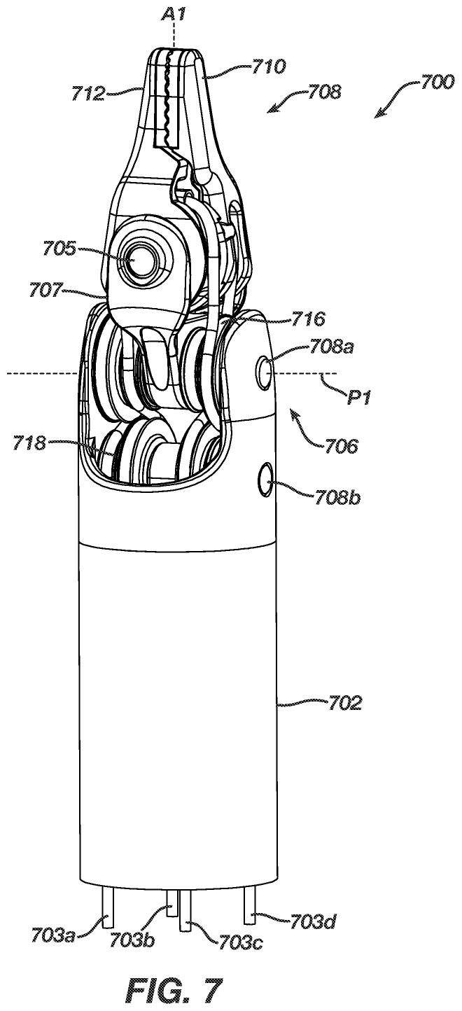

FIG. 7 shows isometric view of illustrative surgical tool 700. Surgical tool 700 contains end effector 704 that incorporates a needle driver and cutting blades. As shown, end effector 704 is operably coupled to wrist 706 via distal clevis 707, which is pivotally engaged with distal wrist axle 708a. A distal plurality of pulleys 716 are also mounted to distal wrist axle 708a, and a proximal plurality of pulleys 718 are mounted to proximal wrist axle 708b. Elongate members 703a-d extend longitudinally within elongate shaft 702 and pass through proximal and distal pluralities of pulleys 716 and 718 before engaging end effector 704. Axes A1 and P1 are the same as those defined with respect to FIGS. 1 and 3-5.

Jaws 710 and 712 of end effector 704 are configured to pivot with respect to one another via rotation about end effector axle 705, which is operably coupled to distal clevis 707. FIG. 7 shows jaws 710 and 712 in a fully closed configuration.

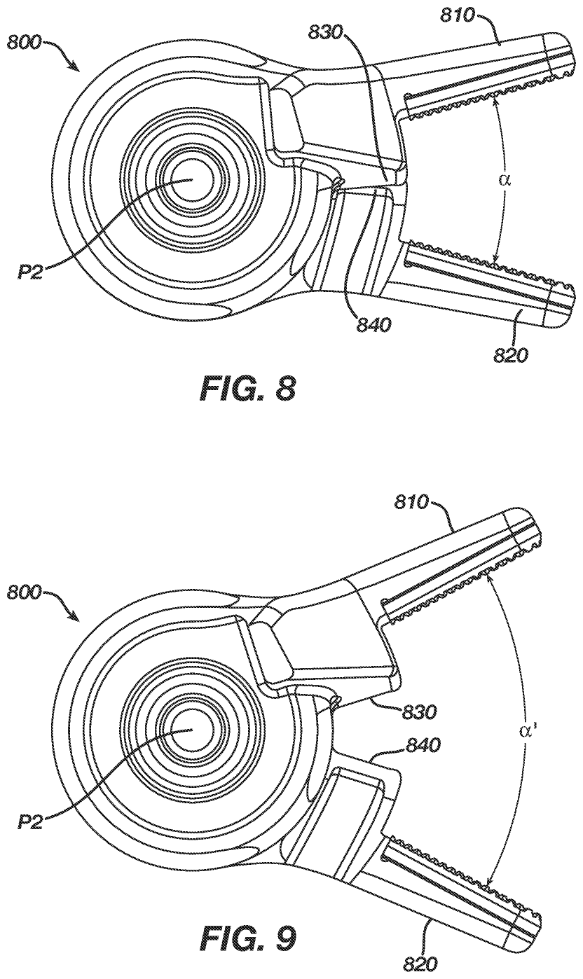

End effector 704 is configured to grasp a needle (or possibly tissue) during a suturing operation when jaws 710 and 712 are closed and to release the needle (or possibly tissue) when jaws 710 and 712 are at least partially open. The extent of opening needed to release a needle during suturing need not necessarily be as wide as that depicted in FIGS. 8 and 9. The closed configuration of FIG. 7 and the partially open configuration of FIG. 8 represent the typical pivoting extremes of jaws 710 and 712 when severing of surgical thread is not desired (i.e., when grasping and releasing a needle). In practice, a much more limited opening of jaws 710 and 712 than that depicted in FIG. 8 can be employed when releasing a needle during a suturing operation, and it can be advantageous to limit the range of opening, as discussed hereinafter.

As shown in FIG. 9, end effector 800 further includes a cutting region with opposing cutting surfaces 830 and 840. As described hereinafter, cutting surfaces 830 and 840 are substantially exposed only when jaws 810 and 820 are opened beyond a predetermined angle α. When jaws 810 and 820 are positioned at an angle less than the predetermined angle α, cutting surfaces 830 and 840 can no longer accept additional surgical thread for cutting. As such, the risk of a premature thread severance is significantly reduced.

Jaws 810 and 820 of end effector 800 may pivot through a first range of angles (α) without exposing cutting surfaces 830 and 840 (see FIG. 8). First range of angles (α) represents the angular positions through which jaws 810 and 820 may pivot between a closed configuration and a partially opened configuration. Jaws 810 and 820 may be articulated within a first range of angles (α) residing between 0) degrees and about 40 degrees, or between about 0 degrees and about 30 degrees, or between 0 degrees and about 25 degrees without exposing cutting surfaces 830 and 840, where an angle of 0 degrees represents a configuration in which jaws 810 and 820 are fully closed.

Jaws 810 and 820 may be articulated through a second range of angles (α′) to open a gap between cutting surfaces 830 and 840 (see FIG. 9). The gap defined between cutting surfaces 830 and 840 when jaws 810 and 820 are articulated within a second range of angles (α′) residing between about 25 degrees and about 45 degrees, or between about 30 degrees and about 45 degrees, or between about 25 degrees and about 40 degrees, or between 30 degrees and about 40 degrees, where 0 degrees represents a configuration in which jaws 810 and 820 are fully closed against one another. The second range of angles (α) includes angles greater than those in the first range of angles (α).

In various embodiments, the first range of angles (α) represents the extent of jaw articulation over which surgical tool 800 is typically utilized for performing a suturing operation, with an angle of substantially 0 degrees being employed when grasping a suturing needle and an angle up to about 25 degrees, or up to about 30 degrees, or up to about 40 degrees being employed when the suturing needle is released. More particular, the suturing needle may be released from jaws 810 and 820 at an angle much less than that at which the gap between cutting surfaces 830 and 840 becomes defined, such as any angle above 0) degrees and up to about 20 degrees or any angle above 0 degrees and up to about 15 degrees. Likewise, the second range of angles (α′) corresponds to the extent of jaw articulation over which surgical tool 800 can receive surgical thread for severing by placing the surgical thread in the gap defined between cutting surfaces 830 and 840 and then decreasing the angular separation until cutting surfaces 830 and 840 slidingly engage one another once again.

In some embodiments, cutting surfaces 830) and/or 840 may be fabricated integrally as a one-piece construct with a jaw body rotatably coupling corresponding jaw(s) 810 and/or 820 to end effector axle P2, thereby allowing articulation to take place. In other embodiments, cutting surfaces 830 and/or 840 and a corresponding jaw body may be fabricated as separate components that are configured to mate together so that they can pivot in tandem with one another as illustrated in Patent Application Publication 2019/0105032 previously incorporated by reference herein.

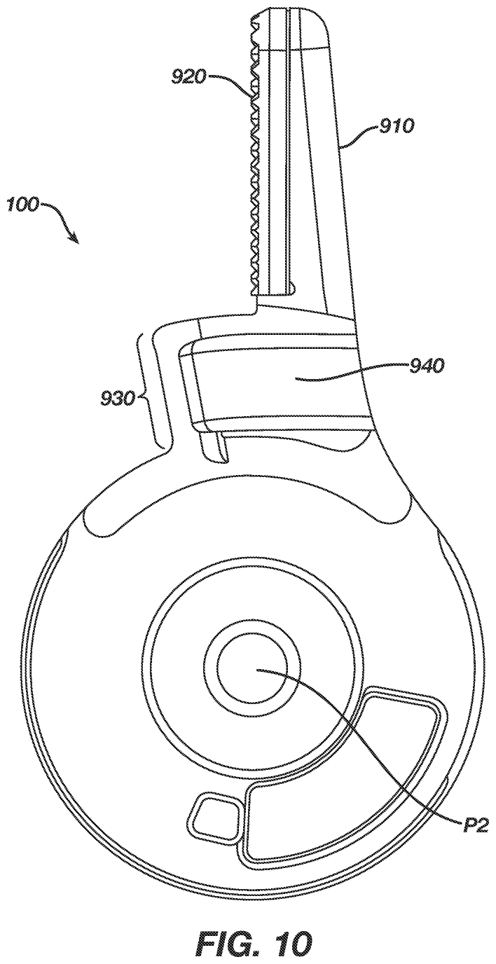

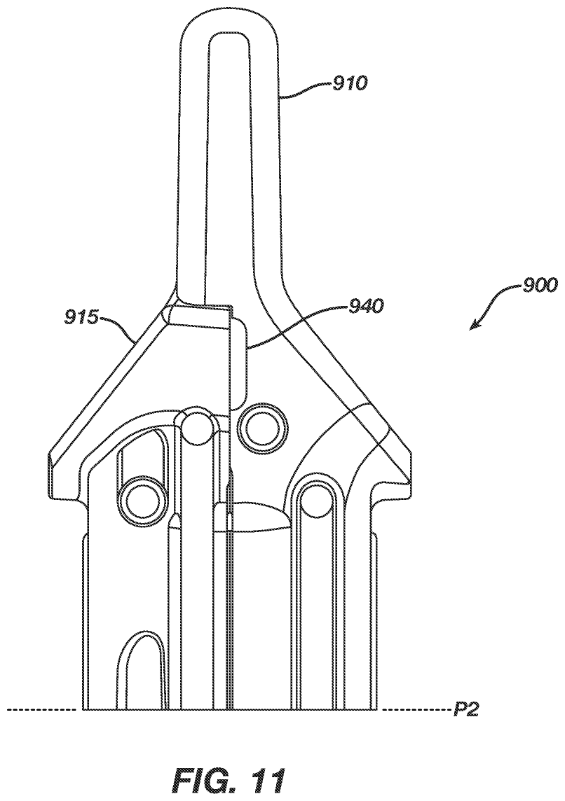

FIG. 10 shows a cross-section of end effector 900 that pivots about axis P2. End effector 900 has a jaw 910 that may include a gripping portion 920 for grabbing, holding, and manipulating tissue, surgical thread, or a suture needle. The surface of gripping portion 920 may be textured, grooved, or otherwise customized to improve the grip. End effector 900 further includes a cutting region 930 that has a sharp inner edge for severing surgical thread. As described above, cutting region 930 is occluded while the jaws of end effector 900 operates within a first range of angles (α) and becomes exposed when the jaws of end effector 900 begin to operating within a second range of angles (α′). Additionally, end effector 900 includes a recessed region 940 that is in close proximity to cutting region 930. With the interior being referenced as coming out of the page and the exterior being referenced as going into the page, recessed region 940 may begin on the same interior plane as cutting region 930 between 0.1 mm and 0.4 mm adjacent to cutting region 930. Recessed region 940 may then taper at an angle of 20 degrees to 60 degrees exterior before forming a new plane approximately 0.1 mm to 0.4 mm exterior to but parallel with the plane of the cutting region. The new plane created by recessed region 940 may continue all the way circumferentially through each respective jaw 910 and 915 and creates an opening exiting out of end effector 900, as best seen in FIG. 11.

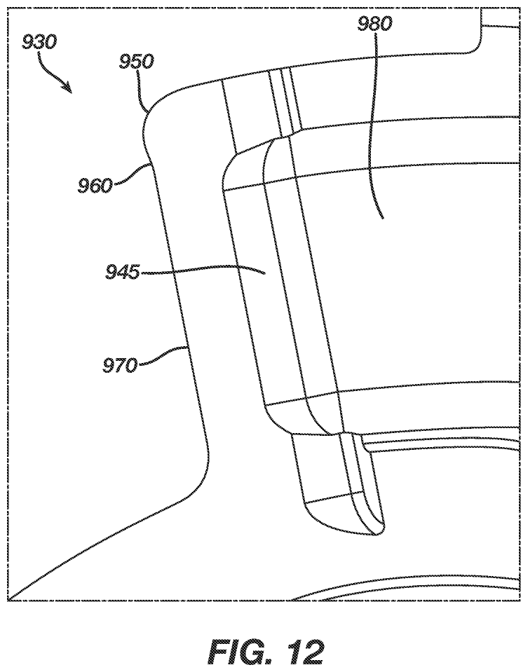

FIG. 12 is an enlarged view of the cutting region 930, and recess region 940, previously shown in FIG. 10. Cutting region 930 may include a substantially flat section followed by several curved sections designed to trap the element being cut within the cutting region. Flat section 970 has a sharp inner edge to act as a blade when cutting tissue or thread and may make up fifty to ninety percent of the total length of cutting region 930. Distal to flat section 970 is a curved concaved section 960, which may also include a sharp inner edge to act as a blade. Concaved section 960 has a radius of curvature between 0.15 mm and 0.35 mm and makes up approximately one to nine percent of the total length of cutting region 930. Distal to concaved section 960 is a convex section 950. Convex section 950 curves in the opposite direction of concave section 960. Convex section 960 may also include a sharp inner edge to act as a blade. Convex section 960 has a radius of curvature between 0.15 mm and 0.35 mm and makes up approximately five to thirty percent of the total length of cutting region 930. While only one jaw is shown in FIG. 12, both jaws 910 and 915 of end effector 900 (shown in FIG. 11) may include cutting region 930. Likewise, jaws 910 and 915 of end effector 900 may also each include a recessed region 940, as previously described. An enlarged illustration of recess region 940 can be seen in FIG. 12. Recess region 940 may include a taper section 945 that begins on plane with cutting region 930 and terminates in recess section 980.

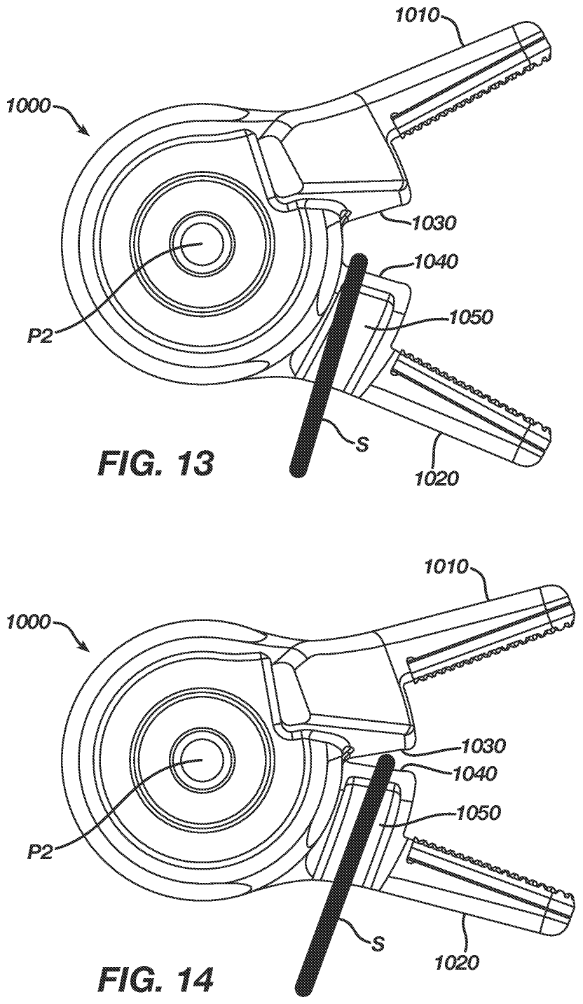

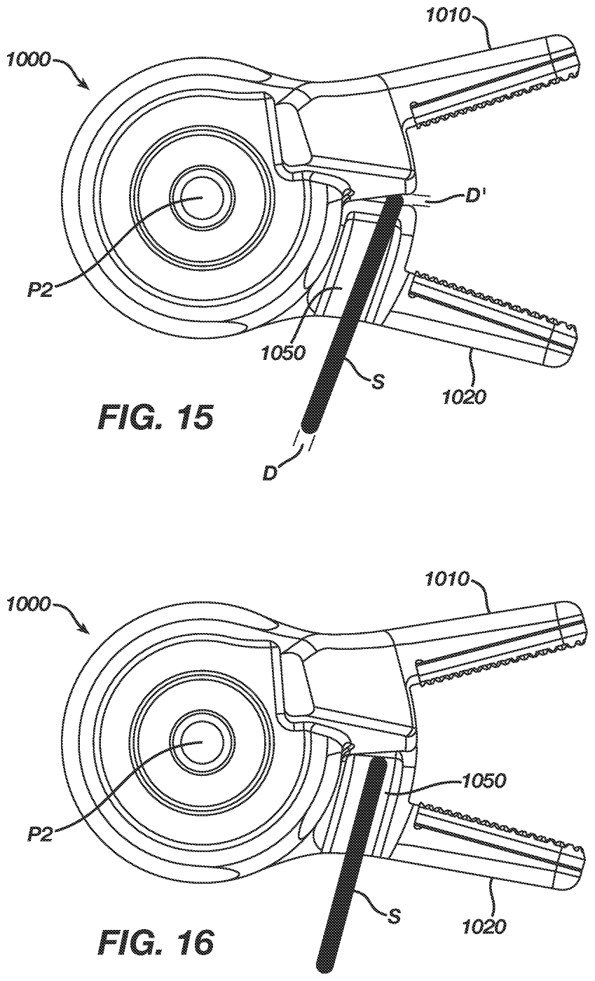

In FIGS. 13-16 an operational sequence of cutting a surgical thread can be observed. When ready to sever the surgical thread S, the user opens jaws 1010 and 1020 beyond the predetermined angular region as previously described, exposing the cutting region of end effector 1000. The user then positions surgical thread S into the cutting region between cutting surfaces 1030 and 1040, as seen in FIG. 13. Once surgical thread S is in position, the user would then begin to close jaws 1010 and 1020 towards each other. As jaws 1010 and 1020 begin to close and contact surgical thread S, in some instances, surgical thread S may not immediately begin to sever but instead begin to slide along the cutting surfaces 1030 and 1040 towards the distal end of end effector 1000 as seen in FIG. 14. Surgical thread S will continue to slide along cutting surfaces 1030 and 1040 until it reaches the point where the convex and concave curvatures of each respective cutting surfaces 1030 and 1040 meet. At this point in the closure of jaws 1010 and 1020 the distance D between the convex portion of each respective cutting surface 1030 and 1040 is smaller than diameter D′ of surgical thread S. Accordingly, because the diameter D′ of surgical thread S is larger than the opening or distance D between the convex portion of each respective cutting surface 1030 and 1040, the surgical thread stops sliding along the cutting surface and begins to be severed by cutting surface 1030 and 1040 as shown in FIG. 15. As the jaws 1010 and 1020 continue to close surgical thread S continues to be severed until the cutting surfaces 1030 and 1040 have completely severed surgical thread S. Once surgical thread S is completely severed the severed ends drop into the recessed region 1050 of each respective jaw 1010 and 1020 as shown in FIG. 16. In the event that jaws 1010 and 1020 continue to close after severing surgical thread S, they will pass over surgical thread S, now in the recess region 1050, and not trap or bind surgical thread S between Jaws 1010 and 1020. After completing the cutting of surgical thread S, the user can move the instrument, in which case surgical thread S will be free to fall out of the recessed region 1050. Alternatively, the user may grab surgical thread S with a different instrument and pull surgical thread S out of the recessed region, even if jaws 1010 and 1020 are in the fully closed position.

The innovations described herein include various aspects, including one or more of the following:

A. A surgical tool that may comprise: an elongate shaft having a proximal end and a distal end; an end effector operably coupled to the elongate shaft at the distal end, the end effector comprising: an end effector axle; a first jaw and a second jaw rotatably mounted to the end effector axle; a first cutting body on the first jaw having a first flat portion, a first concave portion and first convex portion wherein the first flat portion is proximal to the first concave portion and the first concave portion is proximal to the first convex portion; and a second cutting body on the second jaw having a second flat portion, a second concave portion and a second convex portion wherein the second flat portion is proximal to the second concave portion and the second concave portion is proximal to the second convex portion.

B. A surgical tool that may comprise: an elongate shaft having a proximal end and a distal end; an end effector operably coupled to the elongate shaft at the distal end, the end effector comprising: an end effector axle; a first jaw and a second jaw rotatably mounted to the end effector axle; a first cutting body adjacent to the first jaw having a first flat portion, a first concave portion and first convex portion wherein the first flat portion is proximal to the first concave portion and the first concave portion is proximal to the first convex portion; and a second cutting body adjacent to the second jaw having a second flat portion, a second concave portion and a second convex portion wherein the second flat portion is proximal to the second concave portion and the second concave portion is proximal to the second convex portion.

Each of surgical tools described above as A, and B may each independently have one or more of the following additional elements in any combination:

Element 1: wherein the first flat portion is adjacent the first concave portion, the first concave portion is adjacent to the first convex portion, the second flat portion is adjacent the second concave portion, and the second concave portion is adjacent the second convex portion.

Element 2: wherein the first flat portion is more than seventy-five percent the total length of the first cutting body.

Element 3: wherein the first convex and first concave portions have the same radius of curvature.

Element 4: wherein the first convex portion is more than three times the length of the first concave portion.

Element 5: wherein the second flat portion is more than seventy-five percent the total length of the second cutting body.

Element 6: wherein the second convex portion is more than three times the length of the second concave portion.

Element 7: wherein the first jaw has a first recessed portion adjacent to the first cutting body.

Element 8: wherein the second jaw has a second recessed portion adjacent to the second cutting body.

Element 9: wherein the first recessed portion extends circumferentially through the end of the first jaw.

Element 10: wherein the second recessed portion extends circumferentially through the end of the second jaw.

Element 11: wherein a first open configuration is defined by an angular separation between the first and second jaws over a first range of angles (α), and a second open configuration is defined by an angular separation between the first and second jaws over a second range of angles (α′), the second range of angles being larger angles than the first range of angles.

Element 12: wherein the first and second cutting bodies are exposed, and a gap is defined therebetween when the first and second jaws are angularly separated by about twenty-five degrees to about forty degrees.

By way of non-limiting example, exemplary combinations applicable to A, and B include: the surgical tool of A or B in combination with elements 1; 1 and 2; 1, 2 and 3; 1 and 4; 1, 4 and 5; 1, 4, 5, and 6; 7; 7 and 8; 7 and 9; 7, 9, and 10; 7, 9, 10, and 11; 7, 9, 10, 11, and 12; 6 and 11; 6, 11, and 12; 1-6 and 11; 1-6, 11, and 12; 11 and 12.

Unless otherwise indicated, all numbers expressing quantities and the like in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

While various systems, tools and methods are described herein in terms of “comprising” various components or steps, the systems, tools and methods can also “consist essentially of” or “consist of” the various components and steps.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items.

What is claimed is the following:

1. A surgical tool comprising:
an elongated shaft having a proximal end and a distal end;
an end effector operably coupled to the elongated shaft at the distal end, the end effector comprising:
an end effector axle;
a first jaw and a second jaw rotatably mounted to the end effector axle;
a first cutting body on the first jaw having a first flat portion, a first concave portion and first convex portion wherein the first flat portion is proximal to the first concave portion and the first concave portion is proximal to the first convex portion; and
a second cutting body on the second jaw having a second flat portion, a second concave portion and a second convex portion wherein the second flat portion is proximal to the second concave portion and the second concave portion is proximal to the second convex portion.

2. The surgical tool of claim 1, wherein the first flat portion is adjacent the first concave portion, the first concave portion is adjacent to the first convex portion, the second flat portion is adjacent the second concave portion, and the second concave portion is adjacent the second convex portion.

3. The surgical tool of claim 1, wherein the first flat portion is more than seventy-five percent the total length of the first cutting body.

4. The surgical tool of claim 2, wherein the first convex and first concave portions have the same radius of curvature.

5. The surgical tool of claim 3, wherein the first convex portion is more than three times the length of the first concave portion.

6. The surgical tool of claim 2, wherein the second flat portion is more than seventy-five percent the total length of the second cutting body.

7. The surgical tool of claim 6, wherein the second convex portion is more than three times the length of the second concave portion.

8. The surgical tool of claim 1, wherein the first jaw has a first recessed portion adjacent to the first cutting body.

9. The surgical tool of claim 8, wherein the second jaw has a second recessed portion adjacent to the second cutting body.

10. The surgical tool of claim 8, wherein the first recessed portion extends circumferentially through the end of the first jaw.

11. The surgical tool of claim 10, wherein the second recessed portion extends circumferentially through the end of the second jaw.

12. A surgical tool comprising:
an elongated shaft having a proximal end and a distal end;
an end effector operably coupled to the elongated shaft at the distal end, the end effector comprising:
an end effector axle;
a first jaw and a second jaw rotatably mounted to the end effector axle;
a first cutting body adjacent to the first jaw having a first flat portion, a first concave portion and first convex portion wherein the first flat portion is proximal to the first concave portion and the first concave portion is proximal to the first convex portion; and
a second cutting body adjacent to the second jaw having a second flat portion, a second concave portion and a second convex portion wherein the second flat portion is proximal to the second concave portion and the second concave portion is proximal to the second convex portion.

13. The surgical tool of claim 12, wherein the first flat portion is adjacent the first concave portion, the first concave portion is adjacent to the first convex portion, the second flat portion is adjacent the second concave portion, and the second concave portion is adjacent the second convex portion.

14. The surgical tool of claim 12, wherein the first flat portion is more than seventy-five percent the total length of the first cutting body.

15. The surgical tool of claim 13, wherein the first convex and first concave portions have the same radius of curvature.

16. The surgical tool of claim 14, wherein the first convex portion is more than three times the length of the first concave portion.

17. The surgical tool of claim 13, wherein the second flat portion is more than seventy-five percent the total length of the second cutting body.

18. The surgical tool of claim 17, wherein the second convex portion is more than three times the length of the second concave portion.

19. The end effector of claim 18, wherein a first open configuration is defined by an angular separation between the first and second jaws over a first range of angles (α), and a second open configuration is defined by an angular separation between the first and second jaws over a second range of angles (α′), the second range of angles being larger angles than the first range of angles.

20. The end effector of claim 19, wherein the first and second cutting bodies are exposed, and a gap is defined therebetween when the first and second jaws are angularly separated by about twenty-five degrees to about forty degrees.
